SEQUENCE LISTING (7 sequences)

SEQ ID NO: 1 - protein, 23 aa, Human immunodeficiency virus
  PEPTIDE (1)..(23): Sequence between amino acids 519 and 541 at the N-terminal end of HIV Gp41 protein
AVGIGALFLGFLGAAGSTMGARS

SEQ ID NO: 2 - protein, 16 aa, Drosophila melanogaster
  PEPTIDE (1)..(16): Sequence of the third helix of the homeodomain of the Antennapedia transcription factor
RQIKIWFQNRRMKWKK

SEQ ID NO: 3 - protein, 29 aa, Sendai virus
  PEPTIDE (1)..(29): Internal fusogenic domain of the F1 protein of the Sendai virus
TAALRLGIKLTQHYFGLLTAFGSNFGTIG

SEQ ID NO: 4 - protein, 22 aa, Homo sapiens
  PEPTIDE (1)..(22): Peptide obtained from the VAMP1 protein (synaptobrevin 1)
MMIMLGAICAIIVVVIVIVFFT

SEQ ID NO: 5 - protein, 18 aa, Unknown
  Synthetic construct
RGGRLSYSRRRFSVSVGR

SEQ ID NO: 6 - protein, 13 aa, Human immunodeficiency virus
  MISC_FEATURE (1)..(1): Xaa represents a Cys-acetamidomethyl
XGRKKRRQRRRQC

SEQ ID NO: 7 - protein, 20 aa, Human T-cell lymphotropic virus type 1
  PEPTIDE (1)..(20): Peptide corresponding to sequence 242-261 of the Gp46 protein of the HTLV-1 virus
SPNVSVPSSSSTPLLYPSLA

(19) United States
(12) Patent Application Publication
(10) Pub. No.: US 2011/0256552 A1
(43) Pub. Date: Oct. 20, 2011

(54) METHOD FOR PREPARING A SUBSTRATE FOR IMMOBILIZING A CELL, SAID SUBSTRATE AND USES THEREOF

(76) Inventors: Gérard Deleris, Bordeaux (FR); Sandra Rubio Albenque, Saint Jean D'Illac (FR); Bernard Bennetau, Camblanes et Meynac (FR); Bernard Desbat, Pessac (FR); Frédéric Buffiere, Pessac (FR); Jean-Luc Chagnaud, La Brede (FR)

(21) Appl. No.: 12/450,741

(22) PCT Filed: Apr. 11, 2008
(86) PCT No.: PCT/EP2008/054442

§ 371 Date: Apr. 21, 2011

(30) Foreign Application Priority Data
Apr. 12, 2007 (FR) F07/54424

(51) Int. Cl. G01N 33/53; C07K 1/04; C07H 23/00; C12Q 1/02; C07F 7/18; C12N 11/02; C07K 17/00

(52) U.S. Cl. 435/7.2; 435/177; 530/345; 530/326; 530/324; 530/327; 435/29; 556/445; 536/17.1

(57) ABSTRACT

The present invention relates to a method of preparation of a solid substrate capable of immobilizing at least one cell and/or at least one part of a cell, said method comprising a step consisting of fixing, to said solid substrate, a fusogenic compound capable of being inserted in cell membranes. The present invention also relates to a method for immobilizing at least one cell and/or at least one part of a cell using the solid substrate thus prepared, said solid substrate and its uses in the area of biomedical diagnostics or health monitoring of biological fluids or intended for human or animal use.

### TECHNICAL FIELD

The present invention relates to biochemistry, medicine, biology, and in particular analytical biochemistry and immunoassay. More particularly, the present invention relates to the design of an instrument for analysis and for diagnosis (analysis chip or biosensor) permitting samples of varying nature, and notably biological samples, to be examined. Said analysis chip comprises a substrate, optionally functionalized by an organic matrix, a penetrating agent such as a fusogenic compound capable of being inserted in cell membranes and optionally a cell or part of a cell. The present invention relates to the method of manufacture and the use of said substrate for biomedical diagnosis and/or for health monitoring.

### PRIOR ART

During blood transfusions, a conflict may arise between the donor product and, the recipient organism. Transfusion safety must ensure immediate immunologic compatibility and must also prevent incompatibility of future transfusions; this is essential from the standpoint of public health. Poorly managed transfusions can cause the development of antibodies directed against the surface structures of the erythrocytes (antigen) and thus cause hemolysis, which can lead, if there is a severe immune reaction, to the patient's death. Moreover, it is essential to prevent or monitor any undesirable immunization. This applies quite particularly to fetal-maternal immunizations. Thus, following childbirth for the first time, a woman who does not possess the Rhesus D antigen (RH D− phenotype) on her erythrocytes can develop antibodies directed against this antigen present on the surface of the erythrocytes of her first child (RH D+ phenotype). This immunological reaction occurs during contact between the mother's blood and the child's blood during delivery. In the case of a second pregnancy (if the second child also possesses the Rhesus D antigen (RH D+)) an immunological conflict that is serious for the mother and the fetus may develop, sometimes leading to death of the fetus or problems in the neonate (cyanosis: disorder of oxygenation). Thus, testing to find irregular antibodies acquired secondarily (transfusion, pregnancy, transplant) by an individual is of considerable importance. Thus, to ensure that a blood transfusion goes well, systematic screening for irregular antibodies (S.I.A.) is performed in addition to precise phenotyping.

These investigations are performed in all medical analysis laboratories and at blood transfusion centers. Moreover, immuno-hematologic investigations must be rapid and as reliable as possible.

Since its introduction in 1945, the Coombs test based on hemagglutination (aggregation of red blood cells due to the multifunctionality of the antibodies) is still the reference technique in assays for irregular antibodies. Although the test tube technique was the first to be described, it is gradually being replaced by techniques that are less subjective, more sensitive and more standardized. Gel-based techniques were introduced in 1990 and are currently marketed by the companies Diamed and Ortho. Solid-phase techniques, such as capture-R (Immucor), Ready Screen (Biotest) or Biloba (Scibiex) are also on the market. At present, the gel-based technology dominates the immuno-hematology market. However, it has a major disadvantage because of the difficulties of automation.

Thus, owing to the great increase in blood transfusions in hospital environments and the considerable increase in the number of blood analyses in private laboratories, manufacturers are automating their methods of blood analysis.

In order to improve these systems, it would be particularly advantageous to develop a novel immunological tool offering the qualities of the existing tests while providing as many improvements as possible:
simple, direct and rapid visualization of antigen-antibody binding
great reliability,
the best possible sensitivity,
possibility for miniaturization and automation, permitting multiple tests to be combined in a single analysis,
finally, low cost so that it can compete with similar products, which are generally disposable.

The use of spectral methods might satisfy these various points especially with respect to speed, simplicity and sensitivity. Moreover, direct visualization of the interaction would make it possible to evaluate the constants of affinity of the antibodies for the antigens and would thus permit determination and more accurate quantification of the immunoglobulins.

Nowadays, most of these requirements can be met by using technologies that aim to immobilize biomolecules. The latter then make it possible to detect other target analytes. These analytical systems, known as biosensors, have received much study in the areas of agriculture-food, environment and quite particularly the biomedical field, with DNA chips or protein chips, for example.

The main problem when immobilizing cells on a bioreceptor is conservation of their biological properties. Covalent coupling of one of the surface biomolecules of the cell is rarely employed, because grafting techniques affect cellular viability. Most often the cells are merely adsorbed on the surface of the substrate. Sometimes cell growth is carried out directly on the measuring electrode. Finally, a more complex technique consists of using an antibody directed against a recognized cellular element and binding this antibody to the substrate. This technique is for specific use because of the antibody selected, which determines the type(s) of cells immobilized.

### DESCRIPTION OF THE INVENTION

The present invention makes it possible to solve the technical problems mentioned above for the example of irregular antibodies, but can be adapted to numerous other cases in that it offers a tool that can be described more accurately as an analytical system (bioreceptor) composed of a biological element combined with a solid substrate and a measurement circuit (transducer). The properties of molecular recognition of the biological element impart great selectivity and great affinity for interaction between biomolecules and the target analyte. The latter gives rise to a signal that can be translated by various physicochemical methods into a measurement that can be correlated quantitatively and/or qualitatively with the target analyte, which can be a biomolecular or cellular system.

Thus, the present invention proposes the use of a system of supramolecular inclusion that can penetrate the membranes, in order to immobilize cellular elements. This represents an intermediate technique between simple adsorption and covalent coupling, which has not been achievable hitherto.

The present invention permits simple, direct and rapid visualization of the interaction—binding or nonbinding—between two biomolecules with great reliability, and the best possible sensitivity. The possibility of miniaturization and automation makes it possible to combine multiple tests in a single analysis.

Moreover, the present invention is remarkable in that it is not only useful for investigation of irregular antibodies during blood transfusions, but finds applications in any area of biology, medicine, agricultural-food industry and others, where a cell-based chip can be employed.

The present invention relates firstly to a method for preparing a solid substrate capable of immobilizing at least one cell and/or at least one part of a cell, said method comprising a step consisting of fixing, to said solid substrate, a fusogenic compound capable of being inserted in cell membranes.

“Fusogenic compound” means, within the scope of the present invention, any compound that can become anchored in a phospholipid membrane such as a cell membrane and lead to the immobilization of said membrane and therefore to that of a cell.

Within the scope of the present invention, the fusogenic compound can be selected from nonpeptide fusogenic compounds and peptide fusogenic compounds.

“Nonpeptide fusogenic compound” means any molecule containing neither amino acid, nor amino acid analog and capable of becoming anchored in a phospholipid membrane such as a cell membrane and leading to the immobilization of said membrane. These nonpeptide fusogenic compounds notably include the glycosyl phosphatidylinositol (GPI) unit or the polyisoprene units such as the farnesyl unit (isoprene unit with 15 carbon atoms) or the geranylgeranyl unit (isoprene unit with 20 carbon atoms). In fact, numerous proteins with various functions, ranging from enzymatic catalysis to adhesion, are attached to the outer surface of the plasma membrane of eukaryotic cells by anchoring to a GPI. Alkaline phosphatase possesses, at its C-terminal end, such anchoring with a well defined structure (Ronzon, 2001, Thesis Claude Bernard-Lyon University). Isoprenylation is a posttranslational modification which adds a farnesyl or geranylgeranyl group to a protein having a particular unit in the C-terminal position (Maurer-Stroh and Eisenhaber, 2005, Genome Biology, vol. 6, R55).

“Peptide compound” means, within the scope of the present invention, any molecule constituted of amino acids or of amino acid analogs such as peptides, glycopeptides, lipopeptides, pseudopeptides or peptidomimetics. These peptide fusogenic compounds can be linear or branched, having between 5 and 50 amino acids, notably between 7 and 40 amino acids and, in particular, between 10 and 30 amino acids.

In the peptide sequences of the present invention, the amino acids are represented by their single-letter code but they can also be represented by their three-letter symbol according to the following list:

| | | |
|---|---|---|
| A | Ala | alanine |
| C | Cys | cysteine |
| D | Asp | aspartic acid |
| E | Glu | glutamic acid |
| F | Phe | phenylalanine |
| G | Gly | glycine |
| H | His | histidine |
| I | Ile | isoleucine |
| K | Lys | lysine |
| L | Leu | leucine |
| M | Met | methionine |
| N | Asn | asparagine |
| P | Pro | proline |
| Q | Gln | glutamine |
| R | Arg | arginine |
| S | Ser | serine |
| T | Thr | threonine |
| V | Val | valine |
| W | Trp | tryptophan |
| Y | Tyr | tyrosine |

In a first embodiment of the present invention, the peptide fusogenic compound used is a basic peptide obtained from viral proteins, from transcription factors or from toxins. Several basic peptides derived from viral proteins, from transcription factors and from toxins possess the ability to pass through membranes without changing them (Thoren et al., 2000, FEBS Lett., vol. 482, pages 465-8). This led to the development of the first basic vector of 16 amino acids called Penetratine (international application WO 97/12912). This peptide is derived from the homeodomain of a transcription factor of Antennapedia drosophilia. Its special properties mean that it can be used as a vector for transporting hydrophilic active substances into cells. Several other internalization vectors are also known, such as those described in the international application WO 99/07728.

In a second embodiment of the present invention, the peptide fusogenic compound used is a peptide whose amino acid composition is rich in hydrophobic amino acids, i.e. rich in alanine, isoleucine, leucine, methionine, phenylalanine, tryptophan, tyrosine, valine. Advantageously, the peptide fusogenic compound comprises at least 40%, notably at least 50% and, in particular, at least 60% of hydrophobic amino acids relative to the total number of amino acids in its sequence. Said fusogenic peptides are advantageously peptides derived from signal peptides and, more particularly, from the hydrophobic domain of the latter or from peptide fragments of membrane proteins notably of viruses such as HIV (human immunodeficiency virus), HTLV (human T-cell lymphoma virus), MLV (murine leukemia virus) and herpes virus.

The peptide fusogenic compound according to the present invention is advantageously selected from the group comprising the peptides having the following sequences:
AVGIGALFLGFLGAAGSTMGARS (SEQ ID NO. 1 in the appended sequence listing) corresponding to the sequence between amino acids 519 and 541 at the N-terminal end of HIV Gp41 protein,
RQIKIWFQNRRMKWKK (SEQ ID NO. 2 in the appended sequence listing) corresponding to the sequence of the 3^{rd} helix of the homeodomain of the Antennapedia transcription factor, a peptide that is also known by the name Penetratine,
TAALRLGIKLTQHYFGLLTAFGSNFGTIG (SEQ ID NO. in the appended sequence listing) corresponding to the sequence of the internal fusogenic domain of Fl protein of the Sendai virus, peptide SV201 in the article of Ghosh et al., 2000, Biochem., vol. 39, pages 11581-92;
MMIMLGAICAIIVVVIVIVFFT (SEQ ID NO. 4 in the appended sequence listing) corresponding to the transmembrane sequence of the Vamp protein of the SNARE system involved in exocytosis,
RGGRLSYSRRRFSVSVGR (SEQ ID NO. 5 in the appended sequence listing) corresponding to a peptide derived from protegrins and, more particularly, to peptide SM1738 in the international application WO 99/07728,
C(Acm)GRKKRRQRRRQC with C(Acm)=Cys-acetamidomethyl (SEQ ID NO. 6 in the appended sequence listing) corresponding to the basic internalization peptide TA-T of HIV,

derivatives thereof and fragments thereof.

The present invention envisages using derivatives of the fusogenic peptides defined above. “Derivative of the fusogenic peptides” means peptides having 60%, 65%, 70%, 75%, 80%, 85%, 90% and/or 95% of identity with the sequences of the preferred fusogenic peptides given above. The derivatives of the fusogenic peptides can also have, relative to the sequences of the fusogenic peptides given above, at least one additional amino acid in the C-terminal portion and/or in the N-terminal portion, a posttranslational modification and/or a chemical modification, in particular a glycosylation, amidation, acylation, acetylation, methylation, as well as peptides bearing a protecting group, which can prevent their degradation.

The derivatives of the fusogenic peptides can also be those in which one or more amino acids are selected from the group comprising enantiomers, diastereoisomers, natural amino acids of conformation, beta amino acids, substituted alpha amino acids, rare amino acids notably hydroxyproline, hydroxylysine, allohydroxylysine, 6-N-methyllysine, N-ethylglycine, N-methylglycine, N-ethylasparagine, alloisoleucine, N-methylisoleucine, N-methylvaline, pyroglutamine, aminobutyric acid and synthetic amino acids notably ornithine, norleucine, norvaline, cyclohexyl-alanine and omega-amino acids. The derivatives of the fusogenic peptides also cover, according to the invention, the retropeptides and the retroinversopeptides, as well as the peptides in which the side chain of one or more amino acids is substituted with groups that do not modify the fusogenic activity of said fusogenic peptides.

The preferred fragments of fusogenic peptides advantageously have more than 5 amino acids, notably more than 10 amino acids or even more than 15 amino acids.

It is clear that the derivatives and the fragments of fusogenic peptides that can be used within the scope of the present invention must also display fusogenic activity. It will be easy for a person skilled in the art to verify the presence of said activity, notably by using the technique described in the experimental section given below.

The fusogenic peptides, derivatives thereof and fragments thereof can be natural products, recombinant products obtained according to techniques of molecular biology and of genetic engineering well known by a person skilled in the art or can be synthesized chemically according to techniques such as solid-phase or liquid-phase synthesis, also well known by a person skilled in the art.

Within the scope of the present invention, the solid substrate on which the fusogenic compound is fixed is notably an inorganic solid substrate. Advantageously, the solid substrate according to the present invention is selected from the group comprising glasses, quartz, silicas, ceramics (for example, of the oxide type), metals (for example, aluminum, chromium, copper, zinc, silver, nickel, tin or gold) and semiconductors (for example, silicon, germanium, ITO). In another variant of the invention, the solid substrate or the surface of said solid substrate is of an organic material such as a polymer or a resin including nylon, polyethylene glycol, polycarbonates, polyfluoro polymers or composites.

Said solid substrate can be in various forms and of variable size. As examples, and nonexhaustively, it can be in the form of laminas, microplates, particles, beads or microchannels of the capillary type. These various types of substrate can have sizes varying from some hundreds of micrometers to several centimeters.

In a first embodiment of the present invention, the solid substrate has a surface bearing functional groups (designated “functionalized surface” hereinafter). Advantageously, these functional groups are selected from hydroxyl groups, radical entities, alcohol, amine or thiol functions. This functionalization can be intrinsic to the nature of the material at the surface of the solid substrate employed. Alternatively, this functionalization can be obtained by cleaning said surface using at least one solvent, detergent, radiation or oxygen plasma or any other method permitting the formation of functional groups as previously defined.

In a first variant of the present invention, the fusogenic compound can be bound directly to the solid substrate, functionalized or not.

In a second variant of the present invention, binding between the fusogenic compound and the functibnalized or unfunctionalized solid substrate is indirect and is achieved by means of a linking agent. In this embodiment, a functionalized solid substrate is advantageously selected. In this second variant, at least one linking agent is grafted beforehand to the surface of said solid substrate. This (or these) linking agent(s) then provide(s) the fixation of the fusogenic compounds on the solid substrate and notably on solid substrates whose surface is inorganic. A person skilled in the art knows various linking agents that can be used. As an example and nonexhaustively, this embodiment corresponds to the case of a substrate covered with a thin layer of a siloxane polymer of the polyethylene/polyethylene glycol type or of polylysine (D or L) enabling the fusogenic compound to be fixed. “Polymer” means a repetition of a certain number of monomer units advantageously between 2 and 30.

In a quite particular variant of the present invention, the indirect fixation of the fusogenic compound on the solid substrate is achieved by means of a self-assembled organized monolayer of one or more organic or organometallic compounds (Si, Sn, Ge) possessing an alkyl chain terminated by a functional group. These functional groups are, for example, a hydroxyl, an amino, a carboxyl, a halogen or a thiol as well as their modified forms, notably activated or protected forms.

Ulman described, in Chem. Rev., 1996, vol. 96, pages 1533-54, the formation of self-assembled monolayers of functionalized alkyltrichlorosilanes. Their use for the fixation of nucleic acids was described in the patent application FR 2 804 129. Thus, within the scope of the present invention, the self-assembled organized monolayer comprises one or more organosilicon compounds corresponding to the following formula I:

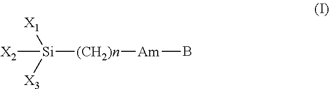

in which
n is between 3 and 40,
m is equal to 1,
X₁, X₂, X₃, which can be identical. or different, are selected from the group comprising saturated, linear or branched C₁ to C₆ alkyl groups, and hydrolyzable groups, at least one of X₁, X₂ and X₃ representing a hydrolyzable group,
A represents the group —O—(CH₂CH₂O)ₖ—(CH₂)ᵢ— in which k is between 1 and 100, and i is an integer greater than or equal to 0
m being equal to 1 and k≧1
if i=0, then B represents a group —R₁, —COR₁, —COOR₁, —CONR₁R₂,
if i≧1, then B represents a group —OR₁, —OCOR₁, —NR₁R₂, —COOR₁, —CONR₁R₂, —SR₁ or a halogen atom.

R₁ and R₂, which may be identical or different, represent a hydrogen atom, a hydrocarbon chain optionally substituted, saturated or unsaturated and linear or branched comprising 1 to 24 carbon atoms or an aromatic group.

When B represents a group —OR₁, —OCOR₁ or —COOR₁, regardless of the values of i and when k≧1 it is understood that B can represent any group resulting from the protection of a hydroxyl or carboxylic acid function such as the protecting groups described in Protective groups in organic system (T. W. GREENE et al., 2nd ed., Wiley Interscience), for example a cyclic protecting group.

Advantageously, i is between 0 and 100, notably between 0 and 50, in particular between 0 and 10 and, more particularly, i is 0 or 1.

In the sense of the present invention, “hydrolyzable” means any group capable of reacting with an acid in an aqueous medium so as to give the compounds X₁H, X₂H or X₃H, X₁, X₂, X₃ being as defined in formula I. According to an advantageous embodiment, said hydrolyzable group is selected from the group comprising the halogen atoms, the —N(CH₃)₂ group and the groups —OR′, R′ being a saturated, linear or branched C₁ to C₆ alkyl group.

“Halogen” means chlorine, bromine or iodine, as well as fluorine.

Surprisingly, to be used within the scope of the present invention, it is suitable for the organosilicon compound or compounds of formula I to have an ethylene glycol.

A self-assembled organized monolayer formed on a solid substrate makes it possible to obtain an organic surface that is dense, homogeneous and with parameters that are well defined both chemically and structurally. The formation of this monolayer, obtained owing to the properties of self-assembly of the compounds of formula I for well-defined values of n, m, k, and i is perfectly reproducible for one or every organosilicon compound or for mixtures of several compounds both in terms of quantity and in terms of distribution on the surface of the substrate. This functionalization is stable over time and the grafted molecules display good orientation with respect to the biological molecules. These parameters of distribution of the organosilicons on the solid substrate are determined and monitored by various optical methods such as vibrational imaging, atomic force microscopy, ellipsometry etc.

Moreover, the formation of a very dense self-assembled organized monolayer protects the siloxane bonds against chemical treatments (notably acid or basic), making it possible to carry out various chemical reactions on the surface of the substrate.

The organosilicon compounds of formula I used in the present invention advantageously have very varied functionalities and great reactivity, considering the nature of group A and the variety of terminal groups B that can be used, it being possible of course for said groups B to be modified and functionalized at will according to the reactions of organic chemistry that are well known by a person skilled in the art.

Whether the binding of the fusogenic compound to said solid substrate is direct or indirect, the bonds involved between the peptide compound, the solid substrate and/or the linking agent are selected from covalent, ionic, or electrostatic bonds or any strong chemical interaction without degradation of the siloxane bonds formed between the organosilicon compounds and the solid substrate.

Thus, within the scope of the present invention, the method of preparing a solid substrate capable of immobilizing at least one cell or at least one part of a cell advantageously comprises the following steps:

a) preparation of a solid substrate as defined previously, modified with a self-assembled monolayer comprising at least one organosilicon compound corresponding to formula I as defined previously, said organosilicon compound having at its end a halogen, a hydroxyl, acid or amine function, protected or not, activated or not,

b) optionally, deprotection of the terminal function of said organosilicon compound used in step (a),

c) optionally, in the case when the modified solid substrate bears carboxylic acid terminal functions, activation of said functions,

d) optionally, deprotection of the side chains and of the terminal amine of the peptide fusogenic compound as defined previously,

e) optionally, in the case when the modified solid substrate bears hydroxyl or terminal amine functions, activation of the carboxylic acid terminal function of the peptide fusogenic compound as defined previously,

f) contacting the modified solid substrate obtained in steps (a), (b) or (c) by immersion, for a specified duration, with one or more solutions, in one or more polar solvents, of the peptide compound or compounds as defined previously to be immobilized,

g) optionally, washing of the substrate on which said peptide compound or compounds are immobilized after step (f).

Treatment step (b) can for example be a basic treatment and optionally with ultrasound in order to remove the organosilicon compounds that are only adsorbed on the surface.

Steps (c) and (e) of activation of the carboxylic acid functions can for example be carried out by means of a solution of N-hydroxysuccinimide or of carbodiimide, or any other suitable activation reagent known by a person skilled in the art.

Step (f) is carried out in conditions of temperature from 0 to 70° C. and in a satisfactory range of pressure. In step (f), it is to be understood that, for dissolving the peptide compounds, any solution permitting good solubility of the latter and control of evaporation of the solution can be used. Fixation of the peptide compounds in the course of step (f) can be monitored by various optical or spectroscopic methods (vibrational, visible UV), infrared or Raman imaging, atomic force microscopy, ellipsometry etc.

The peptide fusogenic compounds that are suitable for use in steps (d), (e) or (f) can be used alone or mixed.

During step (g) of washing, notably in a bath of osmosis-treated water, the substrate on which the peptide compounds are grafted can be sonicated, in order to remove the peptide compounds that are only adsorbed on the substrate, without weakening the grafted layer.

The present invention also relates to a method for immobilizing at least one cell and/or at least one part of a cell. This method comprises the following steps:

a′) preparation of a substrate according to a method as defined previously,

b′) preparation of a cellular suspension containing at least one cell or at least one part of a cell,

c′) contacting the solid substrate as prepared in step (a′) by immersion for an indefinite duration in the cellular suspension prepared in step (b′),

d′) at least one washing of the substrate obtained in step (c′) on which said cell or said part of a cell is immobilized.

The preparation of a cellular suspension in step (b′) is advantageously carried out by diluting the cells or parts of a cell in a buffer that is able to preserve the integrity of the cells and of the cell membranes. Prior to said step (b′), the cells can be submitted to various treatments such as centrifugation, washing or concentration. These cells and parts of cells, such as cell membranes, are as described below.

Step (c′) is advantageously carried out in conditions of temperature ranging from 0 to 50° C. and of suitable pressure.

Within the scope of the present invention, “cell” means either a cell of prokaryotic type or of eukaryotic type. Among the eukaryotic cells, the cell can be a yeast, such as a yeast of the genus Saccharomyces or Candida, a mammalian cell, a plant cell or an insect cell. Mammalian cells can notably be tumor cells, cells of a normal somatic line or stem cells. They can be, nonexclusively, red blood cells, osteoblasts, neuronal cells, hepatocytes, muscle cells, lymphocytes or progenitor cells. The cells of prokaryotic type are bacteria, which can be Gram-positive or Gram-negative. Among these bacteria, we may mention, as examples and nonexhaustively, bacteria belonging to the branches of the spirochetes and chlamydiae, bacteria belonging to the families of the enterobacteria (such as Escherichia coli), Streptococcaceae (such as streptococcus), Microccaceae (such as staphylococcus), legionellas, mycobacteria, Bacillaceae and others.

The cells employed within the scope of the present invention can be obtained from a primary cell culture or from a culture of a cell line or from a sample of a fluid such as water or a biological fluid previously extracted from a human or animal body, and said sample can have undergone various prior treatments such as centrifugation, concentration, dilution etc.

“Part of a cell” means, within the scope of the present invention, notably the whole or a portion of the cell membrane in which the fusogenic compound and notably the fusogenic peptide, its derivatives or its fragments as previously defined will become anchored. “Cell membrane” means, within the scope of the present invention, both the phospholipid-rich plasma membrane of eukaryotic cells (also called cytoplasmic membrane, plasmalemma or plasma membrane) and the plasma membrane and the carbohydrate cell wall (containing peptidoglycan) of bacteria or of plant cells.

The parts of cells used within the scope of the present invention can be obtained from cells obtained from a cell culture or from a sample of a fluid as previously defined. A person skilled in the art knows various techniques for obtaining cell membranes, parts of cell membranes, or fractions rich in cell membranes, from cells or from cell cultures, such as the phase partition technique.

The present invention also relates to a solid substrate that can be prepared by the method of preparation according to the invention and/or that can be obtained after immobilization of a cell and/or part of a cell on the latter. The present invention also relates to a diagnostic kit containing at least one solid substrate according to the invention.

In a first embodiment, the present invention relates to a solid substrate as previously defined, on which a fusogenic compound is fixed that is capable of being inserted in cell membranes, said compound being optionally anchored in at least one cell and/or at least one part of a cell as previously described. The substrate according to this first embodiment is remarkable in that it can be preserved before being put to use. Notably it can be frozen, dried or lyophilized. A person skilled in the art knows various techniques of preservation that do not affect the protein structure of the fusogenic compound fixed on said substrate.

In a second embodiment, the present invention relates to a solid substrate as previously defined, on which a fusogenic compound is fixed that is capable of being inserted in cell membranes, said compound being anchored in at least one cell and/or at least one part of a cell as previously described. In this embodiment, it can be called an activated solid substrate, i.e. activated by the cells or parts of cell anchored on said substrate by means of the fusogenic compound.

The substrate according to the present invention as described permits rapid, simple, reproducible, nonspecific and homogeneous immobilization of given cellular elements and can be used for the detection:
of cells that will be immobilized owing to the fusogenic compounds covering the solid substrate (first embodiment above),
of antibodies or of ligands respectively specific to antigens or to receptors present on the cells or parts of cell immobilized on the solid substrate (second embodiment above),
of compounds with potential therapeutic activity by testing them on cells or parts of cell immobilized on the solid substrate, for example cancerous cells (second embodiment above).

The present invention finds particularly interesting application in the field of biomedical diagnosis or health monitoring of biological fluids or those intended for use in humans or animals.

The present invention relates to the use, for the immobilization of biomolecular or cellular elements, of a solid substrate optionally modified by an assembled organized monolayer of one or more organometallic compounds such as organosilicons for example, and to methods of analysis of these biological elements by optical or spectroscopic methods.

Accordingly, the present invention relates to the use of a solid substrate as previously defined within the scope of health monitoring. In fact, the various fusogenic compounds that can be used are inserted in the cell membrane nonspecifically. It is therefore possible to use the substrate bearing the fusogenic compound within the scope of health monitoring for the purpose of verifying the presence or absence of contaminating cells of the bacteria type in a fluid. The present invention makes it possible to concentrate the target to make it detectable. This health monitoring can notably consist of control of the microbiological quality of water or microbiological control in industry.

The present invention relates to the use of a solid substrate as previously defined and/or of a method of immobilization of at least one cell and/or part of a cell on a solid substrate in the investigation of antibodies and/or of ligands respectively specific to antigens or to receptors present on the surface of the cells or parts of cell fixed on said substrate. This embodiment is particularly interesting in the case of the investigation of auto-antibodies when an autoimmune disease is suspected, such as Hashimoto disease, or in the case of investigation of secondarily acquired irregular antibodies notably with a view to blood transfusion. In this particular embodiment, two variants can be envisaged:
either erythrocytes, whose group and/or phenotype are known, are fixed to the solid substrate via the fusogenic compound, and the solid substrate thus activated is put in the presence of a fluid previously taken from an individual such as a mammal whose group and/or phenotype we wish to determine, said fluid being advantageously blood serum,
or erythrocytes from an individual such as a mammal whose blood group and/or phenotype we wish to determine, are fixed to the solid substrate via the fusogenic compound, and the solid substrate thus activated is put in the presence of a fluid containing at least one antirhesus antibody or one directed against the antigen defined.

An antibody on the cell or part of a cell can be detected by various optical or spectroscopic methods (vibrational, visible UV), infrared or Raman imaging, atomic force microscopy, ellipsometry etc.

In fact, all of these different components possess characteristic spectral signatures permitting their detection. It is possible to devise a system for differential detection for observing the presence or absence of biomolecules fixed, specifically, on the cellular elements or of cells themselves anchored on the solid substrate via the fusogenic compound. The application of these detection techniques is a routine task for a person skilled in the art.

As an example, a simple setup for infrared transmission calibrated in the frequency range characteristic of the amide groups will enable the percentage of fixed biomolecules to be found quickly, in comparison with unexposed or unrecognized samples. It is also conceivable, by means of infrared microscopy, to construct an imaging map of the substrate thus treated. It is therefore possible to construct a chip by nanotechnology and thus obtain a plurality of detections to target a very large number of samples.

Other characteristics and advantages of the present invention will become clear on reading the illustrative, nonlimiting examples given below, and referring to the appended drawings.

### BRIEF DESCRIPTION OF THE DRAWINGS

FIG. 1 is a schematic representation of the solid-phase synthesis of peptides 1 and 2. The steps of deprotection and coupling are repeated for each amino acid to be incorporated. One cycle takes from 2 to 3 hours.

FIG. 2 shows the relation between the infrared spectrum of glass materials grafted with two linking agents (organic compounds or linkers C and D) before and after treatment with potassium hydroxide after subtracting the spectrum of the untreated glass.

FIG. 3 is a schematic representation of the indirect fixation of a peptide on a solid substrate via a linker.

FIG. 4 shows the relation between the infrared spectrum of glass materials grafted with the linking agents C^{.} and D then with peptide 1 after subtracting the spectrum of the untreated glass.

FIG. 5 shows the relation between the infrared spectrum of the various materials (glass+compounds C and D (linkers); glass+linker+peptide. 1; glass+peptide 1) with or without ultrasonic treatment after subtracting the spectrum of the untreated glass.

FIG. 6 shows the relation between the infrared spectrum of glass materials grafted with the linking agents C and D and then with peptide 2 after subtracting the spectrum of the untreated glass.

FIG. 7 shows micrographs from photonic microscopy of different glass substrates brought in contact with a cellular suspension and then rinsed. The micrographs in FIGS. 7A and 7B correspond respectively to:

glass functionalized with the coupling agent, namely the linking agents C and D and then with peptide 1,

glass functionalized with the coupling agent, namely the linking agents C and D and then with peptide 2.

FIG. 8 is a schematic representation of the bioreceptor as visualized in the micrograph of FIG. 7B, on which antibodies specific to the membrane antigens of the erythrocytes are fixed.

FIG. 9 shows the micrographs from scanning electron microscopy of different silica substrates brought in contact with a cellular suspension and then rinsed. The micrographs in FIGS. 9A to 9D correspond respectively to:

a silicon substrate oxidized after UV treatment,

a silicon substrate functionalized with linking agents C and D,

a silicon substrate functionalized with linking agents C and D and then with peptide 1,

a silicon substrate functionalized with linking agents C and D and then with peptide 2.

### DETAILED DESCRIPTION OF PARTICULAR EMBODIMENTS

I. Peptide synthesis

Two peptides synthesized in the laboratory were used:
the fusogenic peptide 519-541 corresponding to the NH₂ end of HIV virus Gp41 protein (peptide 2) whose sequence is as follows: AVGIGALFLGFLGAAGSTMGARS (SEQ ID NO: 1 in the appended sequence listing),
the synthetic peptide obtained from Gp46 protein of the HTLV-1 virus (peptide 1) whose sequence (sequence 242-261) is as follows: SPNVSVPSSSSTPLLYPSLA (SEQ ID NO: 7 in the appended sequence listing).

Peptide 1 is particularly interesting. In fact, there is an antibody specifically directed against this peptide 1 (called DB4), which makes it possible to evaluate the conservation of its functionality after grafting.

A solid-phase peptide synthesis (SPPS) according to the Fmoc strategy was used for synthesizing these two peptides.

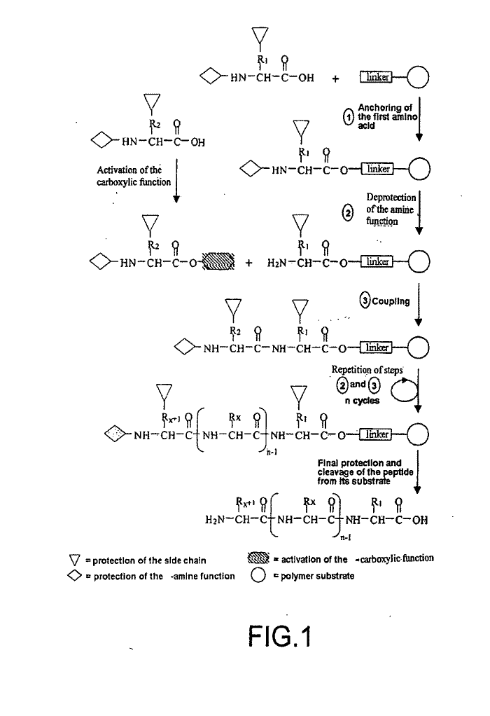

According to the SPPS strategy, synthesis is carried out recurrently from the first amino acid, anchored on the solid substrate by its carboxyl function (step 1, FIG. 1). The 9-fluorenylmethoxycarbonyl Fmoc group (baso-labile) is used for temporary protection of the a-amino function. The liberation of this function is the next step in the synthesis (step 2). The second amino acid, whose α-carboxyl function was previously activated, is coupled to the free α-amino group of the amino acid immobilized on the resin (step 3). Steps 2 and 3 are repeated for each residue to be incorporated.

The syntheses were carried out in the standard conditions of the Fmoc protocol using an automatic synthesizer and by starting the reaction sequence with an amino acid grafted on a Wang type resin. The coupling reactions were carried out in N-methylpyrrolidone (NMP), an aprotic polar solvent that permits a maximum solvation of the peptide-resin assembly. This resin has a degree of presubstitution of about 0.5-0.75 mmol.g⁻¹. It is constituted of beads of polystyrene crosslinked with 1% of divinyl benzene and functionalized with p-benzyloxybenzyl alcohol (linker) which permits binding to the first amino acid.

II. Preparation of the Functionalized Substrate

II.1. Preparation of the Glass Substrate for Grafting

The solid substrate selected is an inorganic glass substrate that has been treated to obtain a surface that is clean and reactive.

In fact, it is necessary to carry out efficient cleaning before any grafting, but this cleaning must not change the nature of the surface. For this, a “Hellmanex II” alkaline detergent was used in order to optimize the cleaning. The surface condition of the material is observed in the infrared by transmission after immersion for 15 minutes at 50° C. in an aqueous solution of 2% Hellmanex. The material is then rinsed with osmosis-treated water, then treatment using jets of osmosis-treated water is applied systematically. Analysis of the plates after this cleaning confirmed disappearance of the bands characteristic of organic contamination (groups CH₃ (2955 and 2875 cm⁻¹) and CH₂ (2920 and 2850 cm⁻¹)).

II.2. Preparation of the Organic Compounds

After conditioning of the glass solid substrate for grafting, organic linkers intended for functionalizing said substrate were synthesized. The use of linkers with a length greater than 17 carbons was selected in order to obtain dense layers leading to good protection of the siloxane bond (Si—O—Si) while preserving good reactivity of the terminal function. Trichlorosilylated molecules, of sufficient length and possessing a functional end, are not available commercially. The synthesis of linkers possessing such properties and notably an alcohol function for the functional end permitting subsequent fixation of a peptide was undertaken.

### a. Synthesis of docos-21-enyl acetate

### Docos-21-en-1-ol

The reaction often used for obtaining a long carbon chain is the coupling of two chains via two sp³ carbons. Heterocoupling reactions are carried out between a Grignard reagent and a halide using a copper-based catalyst, for example LiCuCl₄ or copper-I iodide.

Docos-21-en-1-ol has the formula:

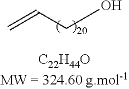

### First step: Formation of 11-bromo magnesium-1-undecene

A solution of 5 g (21.4 mmol) of 11-bromo-1-undecene in 21 ml of THF is added dropwise to a solution of anhydrous THF containing 2.7 g (107 mmol) of magnesium in the form of turnings. This reaction is carried out under an inert atmosphere. The exothermic nature of the reaction is controlled by means of an ice bath. Then 5 ml of dibromoethane is added to the mixture. The reaction is kept active for one hour and the supernatant is removed with a syringe and then put in a flask under inert atmosphere.

### Second Step: Formation of the Lithium Alcoholate

5.4 g (21.4 mmol) of 10-bromo-undecanol is added to a solution of anhydrous THF. The solution is placed under inert atmosphere at −78° C., then using an equal-pressure funnel, 0.71 ml (23.45 mmol) of methyl-lithium is added; then the reaction mixture is allowed to return gradually to room temperature.

### Third step: Formation of docos-21-en-1-ol

0.21 g (1.1 mmol) of copper iodide is added to the solution containing 11-bromo-magnesium-1-undecene cooled to −78° C. The solution containing the lithium derivative is added dropwise using a tube. The mixture is stirred for one hour at this temperature, then for 15 hours at room temperature. The reaction is stopped by adding 40 ml of absolute ethanol; a black precipitate forms. The latter is intensified by adding 3 ml of 10% HCl. After filtration on frit 1, the clear solution obtained is extracted three times with diethyl ether. The ethereal phase that remains is washed with water and then with saturated NaHCO₃ solution. The organic phase is then recovered, dried over MgSO₄ and then concentrated by evaporation of the solvents under reduced pressure. The residue that remains is finally reprecipitated in anhydrous acetone. After filtration on frit 4, 4.86 g (yield=70%) of docos-21-en-1-ol is obtained.

### ii. Docos-21-enyl acetate

The OH end of docos-21-en-1-ol is then acetylated with acetic anhydride in dichloromethane to give docos-21-enyl acetate of formula:

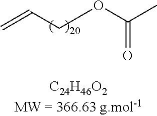

20 ml of anhydrous pyridine is added to 2 g (6 mmol) of docos-21-en-1-ol. After stirring for some minutes, the solution is cooled with an ice bath and 0.7 ml (7.2 mmol) of acetic anhydride is added. After it returns to room temperature, the solution is stirred for 12 hours. The pyridine is then co-evaporated by means of anhydrous toluene, then the residue is immersed in 30 ml of dichloromethane. This solution is washed 3 times with a saturated aqueous solution of sodium hydrogen carbonate. The organic phases are combined after drying over MgSO₄, and the solvents are evaporated under reduced pressure. A white powder is recovered. After purification by chromatography on a silica-gel column with a dichloromethane/methanol mixture (98/2, v/v) as eluent, 1.34 g (yield=62%) of docos-21-enyl acetate, designated organic compound A below, is recovered in the form of a translucent oil which hardens and turns white at room temperature.

b. Synthesis of 1-O-acetyl-10-O-[1-docos-21-enyl]triethylene glycol

### i. Docos-21-ene tosylate

Activation of the hydroxyl end of docos-21-en-1-ol, by the action of tosyl chloride in the presence of triethylamine in dichloromethane at room temperature, enabled us to obtain docos-21-ene tosylate of formula:

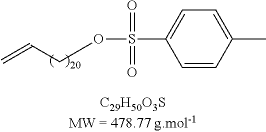

Under nitrogen and at room temperature, 4.6 g (24 mmol) of tosylate chloride diluted in 30 ml of dichloromethane are added to a solution of 4 g (12 mmol) of docos-21-en-1-ol in 4 ml of anhydrous triethylamine and 30 ml of anhydrous dichloromethane. The yellowish mixture is shirred for 12 hours. After filtration on frit 4 and evaporation of the solvents under reduced pressure, the residue is purified by chromatography on a silica-gel column with 100% dichloromethane as eluent. 3.4 g (yield=60%) of docos-21-ene tosylate is recovered.

### ii. 2-(2-(2-(Docos-21-enyloxy)ethoxy)ethoxy)ethanol

83 mg (2.1 mmol) of 60% sodium hydride in oil are added to 1.9 g (12.6 mmol) of triethylene glycol in solution in 30 ml of anhydrous THF. After stirring for 15 minutes under nitrogen, lg (2.1 mmol) of compound 6 in solution in 40 ml of THF is added dropwise. The mixture is then brought to reflux of the THF for 12 hours. After it returns to room temperature, the solution is filtered on frit 4, the solvents are evaporated and the residue is purified by chromatography on a silica-gel column with a dichloromethane/methanol mixture as eluent (98/2, v/v). 0.64 g (yield=67%) of 2-(2-(2-(docos-21-enyloxy)ethoxy)ethoxy)ethanol of the following formula is recovered:

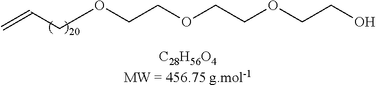

### 2-(2-(2-(docos-21-enyloxy)ethoxy)ethoxy)ethyl acetate

0.5 g (1 mmol) of 2-(2-(2-(docos-21-enyloxy)ethoxy)ethoxy)ethanol is dissolved in a solution of 20 ml of dichloromethane, then 0.26 ml (2.7 mmol) of acetic anhydride is added. The solution is stirred under nitrogen for one hour and then with reflux of the dichloromethane for 12 hours. After it returns to room temperature, the solution is extracted times with 50 ml of dichloromethane. The organic phase is then washed 3 times with a saturated aqueous sodium hydrogen carbonate solution. After drying over MgSO₄ and evaporation of the solvents under reduced pressure of the organic phase, a yellowish paste is recovered. After purification by chromatography on a silica-gel column with a dichloromethane/methanol mixture as eluent (98/2, v/v), 0.45 g (yield=90%) of 1-O-acetyl-10-O-[1-docos-21-enyl]triethylene glycol (or 2-[2-(2-docos-21-enyloxy-ethoxy)-ethoxy]-ethyl acetate) is recovered, designated hereunder as organic compound B of formula:

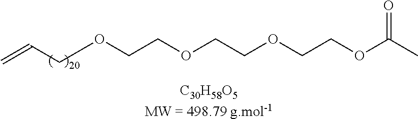

### c. Hydrosilylation of the Organic Compounds A and B

The organic compounds A and B have an unsaturation at one end. This double bond was functionalized by means of chlorosilane to fix, in a second time, these linkers to the glass substrate.

This hydrosilylation was carried out by reaction of trichlorosilane on A and B in toluene in the presence of Kärsted catalyst.

Organic compound A (200 mg (0.5 mmol)) is placed in a Schenck tube that was purged beforehand by switching alternately between a vacuum supply and an argon supply. After adding 2 ml of freshly distilled toluene, the solution is stirred, under argon, until the solid has dissolved completely. Then 300 μl of freshly distilled trichlorosilane is added, plus 2 drops of Kärsted catalyst. The solution, which has become light yellow, is stirred for 2 hours at 40° C. After evaporation under reduced pressure, a raw solid is obtained, and is used as it is in the grafting step. This solid corresponds to 22-(trichlorosilanyl)-docosyl acetate, designated organic compound C hereunder, of formula:

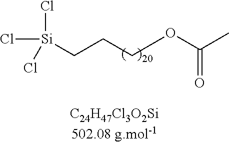

Starting from organic compound B and in the same operating conditions, organic compound D is obtained, corresponding to 1-O-acetyl-10-O-[22-(trichlorosilanyl)-docosyl]triethylene glycol of formula:

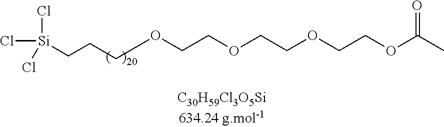

### d. Grafting of the Organic Compounds on the Glass Substrate

After validating the separate grafting of compound C and then of compound D, grafting was carried out by mixing the two types of compounds and using an equimolar mixture with the aim of obtaining a surface having a medium density of sites that are active with respect to the peptide.

Thus, after cleaning, the glass materials are put in a reactor. In this vessel it is possible to dry the material at a controlled temperature, avoiding any organic contamination subsequent to cleaning; the latter often occurs during stove drying. This type of double-walled reactor will also be used for carrying out the silanization step, which must take place under inert atmosphere and at a fixed temperature. This is possible by means of an external cooling system with thermal regulator.

The chlorosilylated compounds C and D (final concentration of each compound 6.10⁻³ M) are dissolved in 90/10 hexane/chloroform mixture. This so-called “silanization” solution is then fed into the reactor containing the materials. Scheme 1 below presents the reaction of grafting compound C on glass.

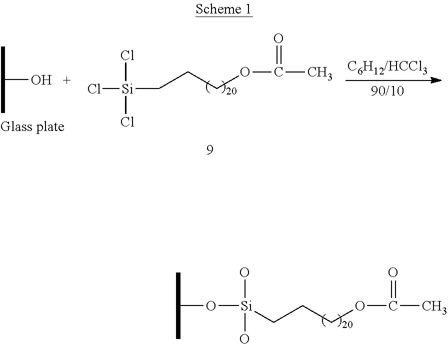

After immersion overnight, the materials are taken out of the vessel and immersed in a bath of osmosis-treated water sonicated for five minutes. This type of treatment removes the organosilicon compounds that. are only adsorbed on the substrate, without weakening the grafted layer.

The next step is deprotection of the OH function by saponification of compounds C and D fixed on the substrate, using alcoholic potassium hydroxide at 0.5 M. The materials are immersed in this KOH solution for 20 minutes. The substrates are then taken out and the impurities are removed by 3 successive treatments of 3 minutes with ultrasound in a bath of osmosis-treated water. The materials are then dried on adsorbent paper. Scheme 2 below corresponds to the reaction of deprotection of the ester function after grafting compound C.

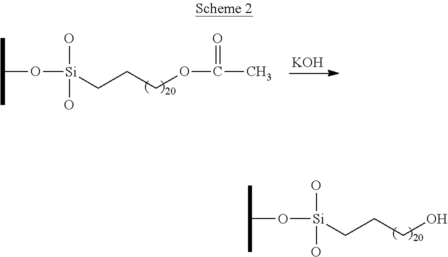

Moreover, in the presence of heterogeneous grafting, KOH penetrates into the grafted. layer, and breaks the bonds binding the organosilicon linkers to the surface. In the case of homogeneous grafting, the molecules of this strong base do not find space between the grafted molecules to reach the surface. The grafting of the inorganic material will therefore remain intact. The use of alcoholic potassium hydroxide therefore also makes it possible to test the homogeneity of the grafting.

In order to validate the grafting of compounds C and D, the variations in hydrophobicity/hydrophilicity of the surface are firstly observed by investigating the contact angle of a water droplet before and after the silanization reaction and after saponification. The results of this analysis of wettability are presented in Table 1.

TABLE 1

| | Number of | | Mean angle obtained |
|---|---|---|---|
| Type | tests | | (°) |
| Untreated glass | 35 | Mean value | 11.66 |
| | | Standard | 2.61 |
| | | deviation | |
| Glass grafted | 30 | Mean value | 74.06 |
| before KOH | | Standard | 2.70 |
| treatment | | deviation | |
| Glass grafted | 30 | Mean value | 51.12 |
| after KOH | | Standard | 2.73 |
| treatment | | deviation | |

A large increase in contact angle after grafting (untreated glass versus treated glass) is obtained, which confirms that a hydrophobic aliphatic layer has formed on the surface. Moreover, after treatment with alcoholic potassium hydroxide, the decrease in this angle shows that the surface has become more hydrophilic after removal of the ester function. This type of analysis is therefore a good indicator of the change in surface condition.

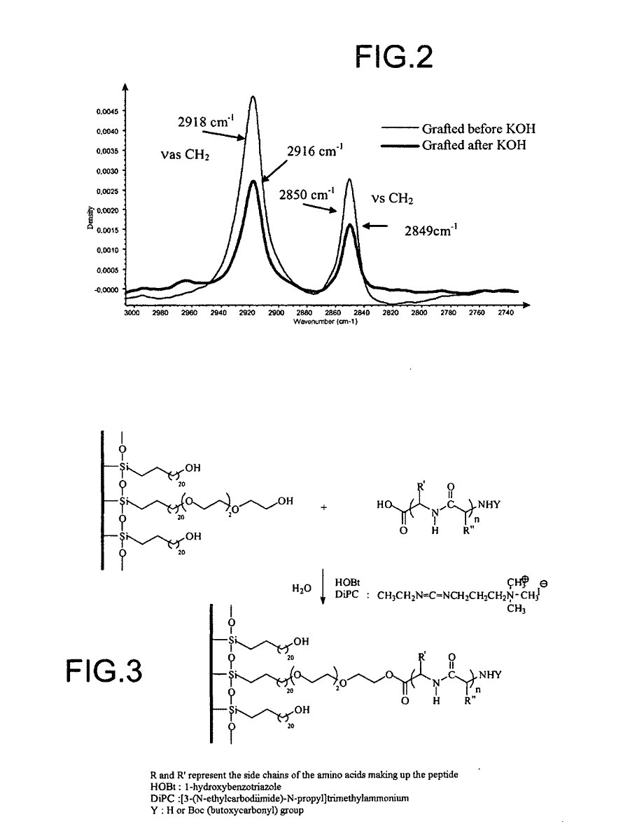

However, in order to obtain more precise information on the grafted elements, transmission infrared analysis was carried out for characterizing the wavenumber corresponding to the CH₂ groups of the aliphatic chains of the grafted compounds C and D. The spectra obtained are presented in FIG. 2.

The analysis confirms the presence of the long aliphatic chain on the surface, by observing the characteristic bands of the CH₂ bonds (νₐₛ=2923 cm⁻¹, νₛ=2853 cm⁻¹). Moreover, the intensity of the bands obtained here (ΔDO=3.10⁻³) is comparable to the simulation carried out with a monolayer of arachidate on glass (ΔDO=2.5.10⁻³). This therefore seems to be a good indication of the formation of a monolayer of compounds C and D on the surface of the substrate. The grafted layer is sufficiently compact and dense to prevent penetration of alcoholic potassium hydroxide into the layer.

FIG. 3 also shows a shift of the spectrum after treatment with alcoholic potassium hydroxide toward low frequencies. Thus, it appears that the presence of the ester group before saponification prevents orientation of the chains relative to one another. After treatment with alcoholic potassium hydroxide, disappearance of this terminal ester results in reorganization of the chains within the layer.

The glass substrate has therefore been functionalized by the two types of compounds C and D so as to obtain a functional surface with low density of active sites.

II.3. Fixation of Peptide 1

Each functionalized substrate is put in a wide-neck tablet bottle, in which the grafting takes place. A magnetized microbar is added to ensure agitation. These tablet bottles are put in the reactor, which is then closed and purged by switching alternatively between a vacuum supply and an argon supply. The substrates are therefore under inert atmosphere.

After adding 8 ml of grafting solvent to each tablet bottle in the reactor, stirring is switched on.

Then the so-called activating solution containing, per unit of substrate, 2 mmol of HOBt and 2 mmol of [3-(N-ethylcarbodiimide)-N-propyl]triethylammonium iodide (DiPC) dissolved, under inert atmosphere, in 1 ml of grafting solvent (osmosis-treated water at 9 g/l of NaCl), is added to the material.

The peptide solution (per substrate, 1 mmol of peptide 1 dissolved under argon in 1 ml of grafting solvent) is added, dropwise, very slowly, with stirring, to the substrate previously immersed in the activating solution. The reaction always follows the reaction protocol shown in FIG. 3.

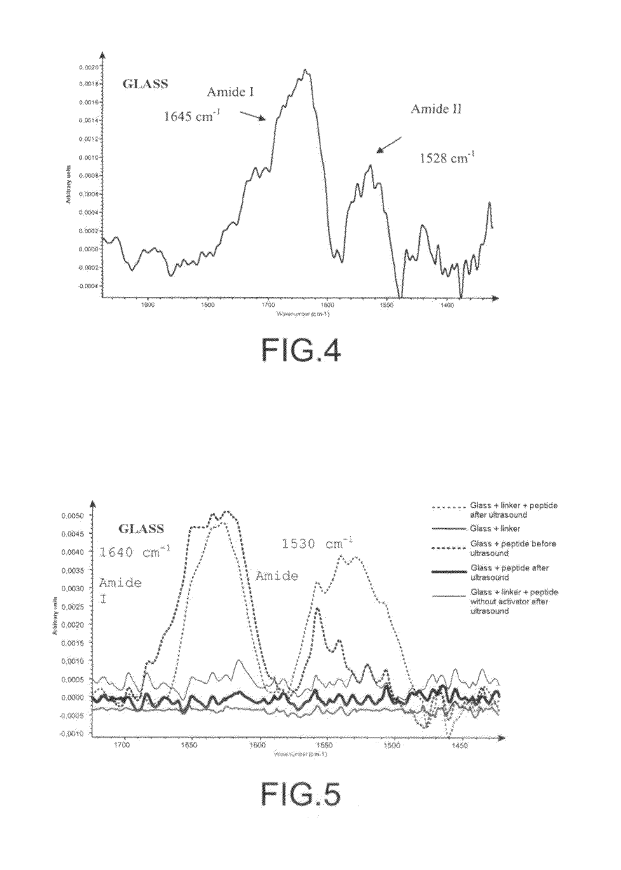

Stirring is maintained for 30 minutes and is then stopped. The substrates are left submerged for hours. The materials are then taken out of the tablet bottles and the molecules that are not fixed covalently are removed with ultrasound and successive treatments of 2 minutes in osmosis-treated water, and are then analyzed as previously. The spectrum obtained is shown in FIG. 4.

Examination of the spectrum reveals the characteristic bands of amide I (ν_{co}) and amide II (combination of ν_{CN} and δ_{NH}). It can therefore be assumed that peptide 1 is present on the surface of the plates.

The glass plates obtained in the various experiments were analyzed by infrared in specular reflection mode and the spectra obtained are presented in FIG. 5.

Thus, analysis of these spectra reveals the presence of peptides only on the plates previously grafted with compounds C and D. This would confirm the hypothesis of covalent grafting rather than simple absorption. Thus, the functional substrates produced permit the covalent fixation of peptides of biological interest.

II.4. Fixation of Peptide 2

Peptide 2 synthesized according to the method described previously was fixed using the same protocol as for peptide 1, except that the osmosis-treated water at 9 g.l⁻¹ of NaCl contained in the solvents for grafting and activation was replaced with hexafluoropropan-2-ol, which makes it possible to dissolve the hydrophobic peptides.

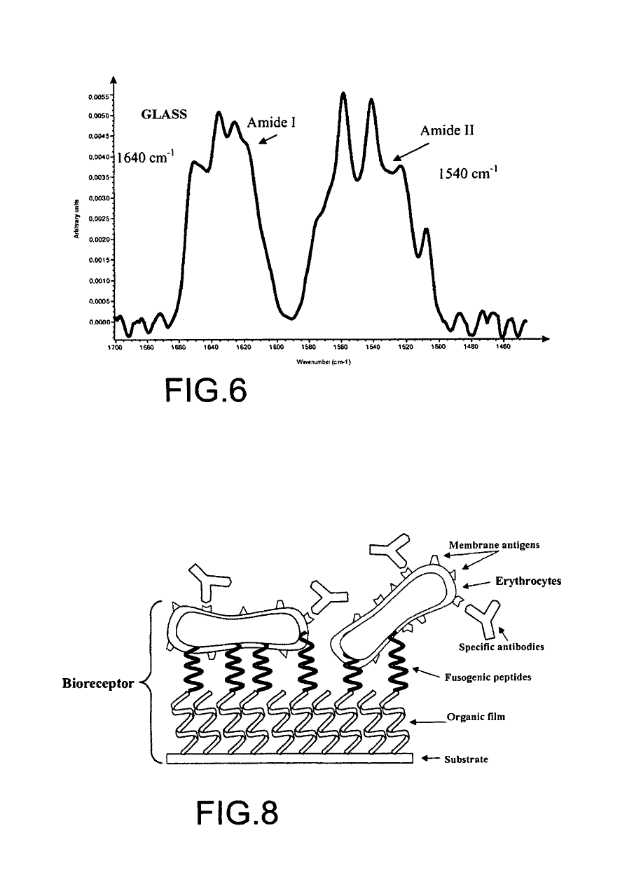

The glass substrates thus obtained are analyzed by infrared in specular reflection mode by the PMIRRAS method (FIG. 6). The characteristic bands of amides I and II confirm that peptide 2 has indeed been grafted on the glass substrate according to the protocol described.

III. Verification of the Biological Properties of the Fixed Peptides

ELISA tests were carried out in order to verify the biological properties and notably the accessibility of the epitope and the specific recognition of antibodies for the fixed peptides.

These tests were notably carried out for peptide 1 specifically recognized by the DB4 antibody, using as negative control an antibody called BF6, directed against a protein of the human complement system and not recognizing peptide 1.

Moreover, the antigen-antibody interaction in the case of peptide 1 and of the DB4 antibody was investigated by transmission infrared spectrometry.

IV. Association of Erythrocytes with the Substrate

The blood used was obtained from the “Etablissement Français du Sang” (French Blood Establishment) and corresponded to a pellet of red blood cells preserved in CPD (citrate-phosphate-dextrose) buffer.

1 ml of this blood is taken, dissolved in 15 ml of PBS 1× buffer and then centrifuged for 5 min at 2500 rev/min. The pellet recovered is then resuspended in the same buffer. This operation, repeated three times, makes it possible to remove a high proportion of the plasma proteins, in order to promote cellular immobilization.

Several glass substrates placed in the wells of a 6-well plate were then immersed in a solution of erythrocytes at 1% in PBS buffer for 1 hour 30 min, stirring gently. The substrates are then held with tweezers and agitated in 3 successive baths of PBS buffer.

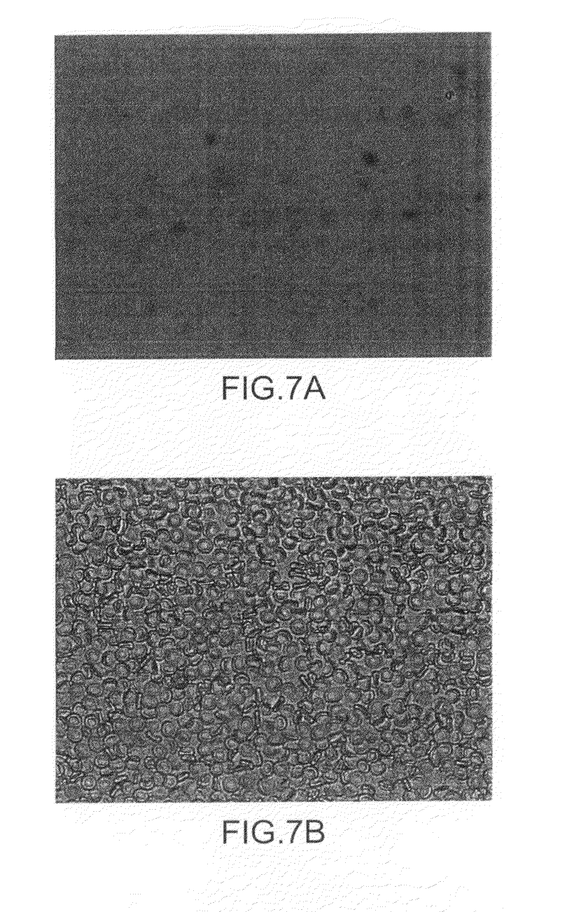

After this rinsing, the glass substrates are preserved in PBS, before being placed between slide and cover slip, to be observed under the photonic microscope NIKON optiphot 2 (Biocom visiol@b). The micrographs obtained (×650 objective 50) are shown in FIG. 7.

This experiment was carried out on:
Glass after treatment with a Hellmanex solution
Glass functionalized with organic compounds C and D,
Glass functionalized with organic compounds C and D and then with peptide 1 (FIG. 7A),
Glass functionalized with organic compounds C and D and then with peptide 2 (FIG. 7B).

No erythrocyte is present on the glass substrates after treatment with Hellmanex solution and glass functionalized with organic compounds C and D. Moreover, no erythrocyte is observed in the micrograph corresponding to the substrate without fusogenic peptide 2 (FIG. 7A). The erythrocytes therefore do not display nonspecific adsorption on the glass, the organic compounds or peptide 1. In contrast, the micrograph obtained for a substrate with the fusogenic peptide 2 (FIG. 7B) shows a high proportion of erythrocytes regularly distributed on the plate. FIG. 8 is a schematic representation of the bioreceptor as visualized in FIG. 7B.

Thus, the fusogenic peptide 2 was able to immobilize the red blood cells on the glass substrate. Moreover, the erythrocytes were undamaged and retained the normal shape of a biconcave disk.

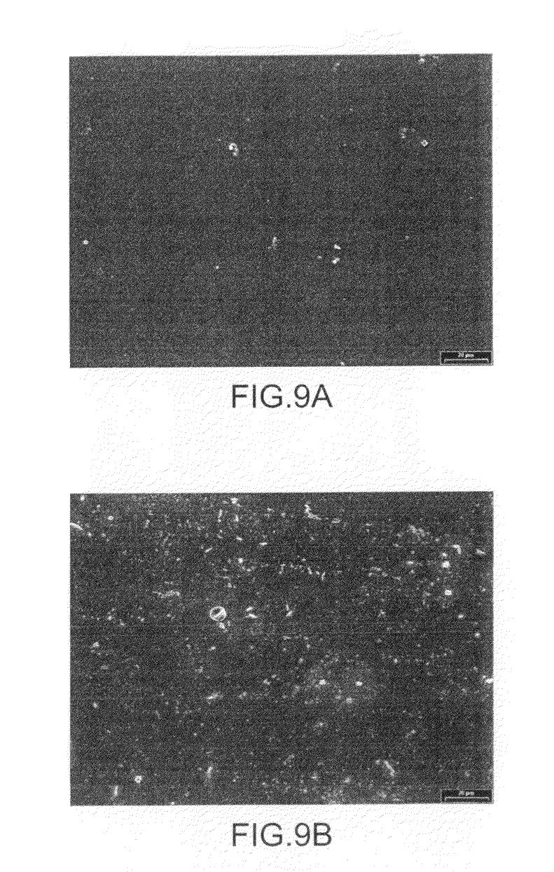

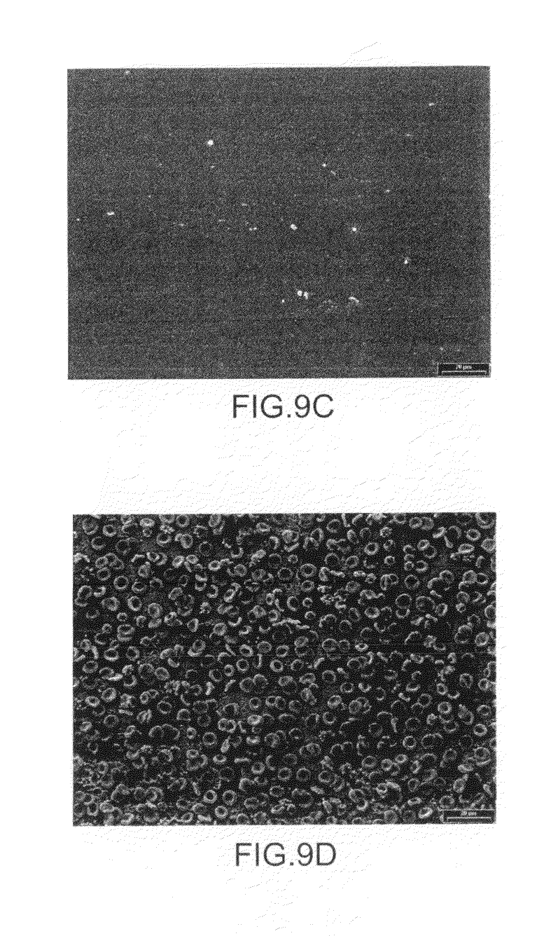

It should be pointed out that results that are entirely similar to those previously presented were obtained using a substrate of oxidized silicon instead of a glass substrate. Thus, experiments identical to those previously described for glass were performed on substrates of oxidized silicon and the results obtained in scanning microscopy are presented in FIG. 9. No erythrocyte is present on the plates without fusogenic peptide 2, i.e. there is no nonspecific adsorption of these cells on the silicon (FIG. 9A), the linking agents C and D (FIG. 9B) or peptide 1 (FIG. 9C).

1-25. (canceled)

26. A method of preparation of a solid substrate capable of immobilizing at least one cell or at least one part of a cell, said method comprising:
fixing, to said solid substrate, a fusogenic compound capable of being inserted in cell membranes.

27. The method of claim 26, wherein said fusogenic compound is a nonpeptide fusogenic compound.

28. The method of claim 26, wherein said fusogenic compound is a peptide fusogenic compound.

29. The method of claim 28, wherein said peptide fusogenic compound is a basic peptide derived from viral proteins, from transcription factors, or from toxins.

30. The method of claim 28, wherein said peptide fusogenic compound is a peptide whose amino acid composition comprises at least 40% of hydrophobic amino acids relative to a total number of amino acids in its sequence.

31. The method of claim 28, wherein said peptide fusogenic compound is selected from among peptides having the following sequences:
AVGIGALFLGFLGAAGSTMGARS (SEQ ID NO. 1 in the appended sequence listing),
RQIKIWFQNRRMKWKK (SEQ ID NO. 2 in the appended sequence listing),
TAALRLGIKLTQHYFGLLTAFGSNFGTIG (SEQ ID NO. 3 in the appended sequence listing),
MMIMLGAICAIIVVVIVIVFFT (SEQ ID NO. 4 in the appended sequence listing),
RGGRLSYSRRRFSVSVGR (SEQ ID NO. 5 in the appended sequence listing),
C(Acm)GRKKRRQRRRQC with C(Acm)=Cys-acetamidomethyl (SEQ ID NO. 6 in the appended sequence listing), and
derivatives thereof and fragments thereof.

32. The method of claim 26, wherein said solid substrate is inorganic.

33. The method of claim 26, wherein said solid substrate is selected from glasses, quartz, silicas, ceramics, metals, and semiconductors.

34. The method of claim 26, wherein said solid substrate or a surface of said solid substrate is of an organic material.

35. The method of claim 26, wherein said solid substrate has a surface bearing functional groups.

36. The method of claim 26, wherein said fusogenic compound is bound directly to said solid substrate.

37. The method of claim 26, wherein a linkage between said fusogenic compound and said solid substrate is indirect.

38. The method of claim 37, wherein said indirect fixation of said fusogenic compound on said solid substrate is achieved using a self-assembled organized monolayer of one or more organic or organometallic compounds possessing an alkyl chain terminated by a functional group.

39. The method of claim 38, wherein said self-assembled organized monolayer comprises one or more organosilicon compounds corresponding to the following formula I: 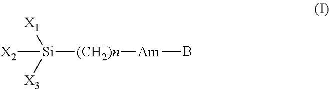
in which
n is between 3 and 40,
m is equal to 1,
X₁, X₂, X₃, which are identical or different, are selected from the group consisting of saturated, linear, or branched C₁ to C₆ alkyl groups, and hydrolyzable groups, at least one of X₁, X₂ and X₃ representing a hydrolyzable group,
A represents the group —O—(CH₂CH₂O)ₖ—(CH₂)ᵢ— in which k is between 1 and 100, and i is an integer greater than or equal to 0,
m being equal to 1 and k≧1
if i=0, then B represents a group —R₁, —COR₁, —COOR₁, —CONR₁R₂,
if i≧1, then B represents a group —OR₁, —OCOR₁, —NR₁R₂, —COOR₁, —CONR₁R₂, —SR₁ or a halogen atom, and
R₁ and R₂ are identical or different and represent a hydrogen atom, a hydrocarbon chain optionally substituted, saturated or unsaturated and linear or branched comprising 1 to 24 carbon atoms or an aromatic group.

40. The method of claim 26, wherein said method further comprises the following steps:
a. preparation of a solid substrate as defined in claim 32, modified by a self-assembled monolayer comprising at least one organosilicon compound corresponding to formula I as defined in claim 39, said organosilicon compound having at its end a halogen, a hydroxyl, acid or amine function, protected or not, activated or not,
b. deprotection of the terminal function of said organosilicon compound used in step (a),
c. in the case when the modified solid substrate bears carboxylic acid terminal functions; the activation of said functions,
d. deprotection of the side chains and of the terminal amine of a peptide compound as defined in claim 28,
e. in the case when the modified solid substrate bears hydroxyl or terminal amine functions, the activation of the carboxylic acid terminal function of a peptide compound as defined in claim 28,
f. contacting the modified solid substrate obtained in steps (a), (b) or (c) by immersion, for a specified duration, with one or more solutions, in one or more polar solvents, of the peptide compound or compounds as defined in claim 28 and after steps (d) or (e), and
g. performing washing said substrate, on which said peptide compound or compounds are immobilized, after step (f).

41. A method for immobilizing at least one cell or at least one part of a cell, said method comprising:
a. preparing a substrate according to a method as defined according to claim 26,
b. preparing a cellular solution containing at least one cell or at least one part of a cell,
c. contacting the solid substrate as prepared in step (a) by immersion for an indefinite duration in the cellular solution prepared in step (b), and
d. performing at least one washing of the substrate obtained in step (c) on which said cell or said part of a cell is immobilized.

42. The method of claim 41, whereih said cell is a eukaryotic cell selected from a yeast, a mammalian cell, a plant cell, or an insect cell.

43. The method of claim 42, wherein said mammalian cell is selected from among red blood cells, osteoblasts, neuronal cells, hepatocytes, lymphocytes, muscle cells, and progenitor cells.

44. The method of claim 41, wherein said cell is a prokaryotic cell such as a bacterium.

45. The method of claim 41, wherein said part of a cell is constituted of the whole or of a portion of a cell membrane.

46. A solid substrate as defined in claim 32, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 42.

47. The solid substrate of claim 46, wherein said fusogenic compound is anchored in at least one cell or at least one part of a cell as defined in claim 42.

48. A diagnostic kit comprising at least one solid substrate as claimed in claim 46.

49. Use of a substrate as claimed in claim 46 for health monitoring.

50. Use of a substrate as claimed in claim 47 or of a method of immobilization of at least one cell or of at least one part of a cell as defined in claim 40 in investigation of antibodies or of ligands respectively specific to antigens or to receptors present on a surface of the cells or parts of a cell fixed on said substrate.

51. The method of claim 27, wherein said nonpeptide fusogenic compound is a glycosyl phosphatidylinositol unit or isoprene units.

52. The method of claim 34, wherein said organic material is a polymer or a resin comprising nylon, polyethylene glycol, polycarbonates, polyfluoro polymers, or composites.

53. The method of claim 33, wherein said method further comprises the following steps:
a. preparation of a solid substrate as defined in claim 32, modified by a self-assembled monolayer comprising at least one organosilicon compound corresponding to formula I as defined in claim 39, said organosilicon compound having at its end a halogen, a hydroxyl, acid or amine function, protected or not, activated or not,
b. deprotection of the terminal function of said organosilicon compound used in step (a),
c. in the case when the modified solid substrate bears carboxylic acid terminal functions, the activation of said functions,
d. deprotection of the side chains and of the terminal amine of a peptide compound as defined in claim 28,
e. in the case when the modified solid substrate bears hydroxyl or terminal amine functions, the activation of the carboxylic acid terminal function of a peptide compound as defined in claim 28,
f. contacting the modified solid substrate obtained in steps (a), (b) or (c) by immersion, for a specified duration, with one or more solutions, in one or more polar solvents, of the peptide compound or compounds as defined in claim 28 and after steps (d) or (e), and
g. performing washing said substrate, on which said peptide compound or compounds are immobilized, after step (f).

54. The method of claim 34, wherein said method further comprises the following steps:
a. preparation of a solid substrate as defined in claim 32, modified by a self-assembled monolayer comprising at least one organosilicon compound corresponding to formula I as defined in claim 39, said organosilicon compound having at its end a halogen, a hydroxyl, acid or amine function, protected or not, activated or not,
b. deprotection of the terminal function of said organosilicon compound used in step (a),
c. in the case when the modified solid substrate bears carboxylic acid terminal functions, the activation of said functions,
d. deprotection of the side chains and of the terminal amine of a peptide compound as defined in claim 28,
e. in the case when the modified solid substrate bears hydroxyl or terminal amine functions, the activation of the carboxylic acid terminal function of a peptide compound as defined in claim 28,
f. contacting the modified solid substrate obtained in steps (a), (b) or (c) by immersion, for a specified duration, with one or more solutions, in one or more polar solvents, of the peptide compound or compounds as defined in claim 28 and after steps (d) or (e), and
g. performing washing said substrate, on which said peptide compound or compounds are immobilized, after step (f).

55. The method of claim 35, wherein said method further comprises the following steps:
a. preparation of a solid substrate as defined in claim 32, modified by a self-assembled monolayer comprising at least one organosilicon compound corresponding to formula I as defined in claim 39, said organosilicon compound having at its end a halogen, a hydroxyl, acid or amine function, protected or not, activated or not,
b. deprotection of the terminal function of said organosilicon compound used in step (a),
c. in the case when the modified solid substrate bears carboxylic acid terminal functions, the activation of said functions,
d. deprotection of the side chains and of the terminal amine of a peptide compound as defined in claim 28,
e. in the case when the modified solid substrate bears hydroxyl or terminal amine functions, the activation of the carboxylic acid terminal function of a peptide compound as defined in claim 28,
f. contacting the modified solid substrate obtained in steps (a), (b) or (c) by immersion, for a specified duration, with one or more solutions, in one or more polar solvents, of the peptide compound or compounds as defined in claim 28 and after steps (d) or (e), and
g. performing washing said substrate, on which said peptide compound or compounds are immobilized, after step (f).

56. A solid substrate as defined in claim 33, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 42.

57. A solid substrate as defined in claim 34, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 42.

58. A solid substrate as defined in claim 35, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 42.

59. A solid substrate as defined in claim 32, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 44.

60. A solid substrate as defined in claim 32, on which a self-assembled organized monolayer as defined in claim 38 is grafted, and on which a fusogenic compound capable of being inserted in cell membranes as defined in claim 26 is fixed, said fusogenic compound being anchored in at least one cell or at least one part of a cell as defined in claim 45.

61. The solid substrate of claim 46, wherein said fusogenic compound is anchored in at least one cell or at least one part of a cell as defined in claim 44.

62. The solid substrate of claim 46, wherein said fusogenic compound is anchored in at least one cell or at least one part of a cell as defined in claim 45.
